(19) United States
(12) Patent Application Publication
(10) Pub. No.: US 2017/0269021 A1
(43) Pub. Date: Sep. 21, 2017

(54) GRAPHENE-POLYMER-ENZYME HYBRID NANOMATERIALS FOR BIOSENSORS

(71) Applicant: Danmarks Tekniske Universitet, Kgs. Lyngby (DK)

(72) Inventors: Qijin Chi, Kgs. Lyngby (DK); Shuang Han, Shenyang City (CN); Arnab Halder, Kgs. Lyngby (DK); Nan Zhu, Søborg (DK); Jens Ulstrup, Klampenborg (DK)

(21) Appl. No.: 15/529,069

(22) PCT Filed: Nov. 18, 2015
(86) PCT No.: PCT/EP2015/076933

§ 371 Date: May 23, 2017

(30) Foreign Application Priority Data
Nov. 27, 2014 (EP) 14195093.1

(51) Int. Cl. G01N 27/327; C12Q 1/54; A61K 9/48; C12Q 1/00

(52) U.S. Cl. CPC G01N 27/3272; C12Q 1/006; C12Q 1/54; C12Y 101/03004; A61K 9/4816; A61K 9/4833; A61K 9/4866; G01N 2333/904

(57) ABSTRACT

The invention relates to a general chemical method for the synthesis of biocompatible hybrid nanomaterials which can be used in the development of new-type enzyme based biosensors. A one-step facile method is presented, in which polyethylenimine (PEI) serves as both a reducing agent for the reduction of graphene oxide (GO) into reduced graphene oxide (RGO) and a biological matrix for accommodation of enzymes.

The invention relates to a general chemical method for the synthesis of biocompatible hybrid nanomaterials which can be used in the development of new-type enzyme based biosensors.

### BACKGROUND

Complete chemical exfoliation of graphite flakes can generate single-layered and water soluble individual graphene oxide (GO) sheets. However, GO is electrically insulating, and if the material is to be used for electronic applications or as electrode materials, the conductivity of the material thus needs to be restored. This can be achieved via chemical or thermal reduction of GO into its reduced form (RGO). Several reducing agents, including hydrazine (N₂H₄),1-dimethylhydrazine,3-sodium borohydride, and hydrogen quinine, have been attempted for reducing GO, showing results with variant efficiency.

Among the above reducing agents, hydrazine is arguably the mostly common used agent. However, hydrazine is not an environmentally and biologically friendly agent. In addition, RGO suspensions prepared by hydrazine have a limited stability ranging from a few hours to days, depending on experimental conditions. CN102850795 discloses an example of a method where hydrazine is used as the reducing agent for reducing GO to RGO. After the reduction of GO to RGO by hydrazine, RGO is mixed with a Ferrocene grafted PEI thereby forming non-covalent bonds between PEI and RGO.

Shanli Yang et al. (Microchim Acta 2013, 180, page 127-135) discloses an alternative method for reducing GO to RGO, wherein a biosensor containing a GO solution is reduced to RGO by immersing a glass carbon electrode into the solution.

Thus, the development of a general chemical route towards the preparation of electrically conductive and biocompatible RGO is of particularly desired for facilitating the use of RGO in biological environments.

### DESCRIPTION OF THE INVENTION

The invention discloses a one-step green reduction and polymeric derivation of graphene oxide (GO), which makes it possible to produce stable and biocompatible reduced GO (RGO) nanosheets. More specifically, here is disclosed a facile environmentally friendly and practical approach to one-step green reduction of graphene oxide by the polymer polyethylenimine (PEI) in an aqueous medium with the simultaneous formation of covalent linkage between the polymer and the RGO.

Disclosed herein is therefore a method for preparing hybrid biofunctional composites comprising reduced graphene oxide (RGO), polyethylenimine (PEI) and an enzyme. The method comprising the steps of A) providing an aqueous solution of graphene oxide (GO); B) reducing the GO by adding PEI to the aqueous solution of GO thereby obtaining an aqueous RGO-PEI solution, and C) mixing the aqueous RGO-PEI solution with an enzyme thereby obtaining the hybrid biofunctional composites.

The RGO and the PEI form covalent bonds and PEI forms a biocompatible matrix electrostatically encapsulating the enzyme inside the matrix. Thus, the enzyme binds non-covalently to the RGO supported PEI.

By the above method is obtained a hybrid biofunctional composite comprising a combination of a conducting element, i.e. RGO, polymer cage/matrix defined by the PEI polymer covalently bound to RGO, and a biological catalyst in the form of an enzyme. The non-covalent bonding between the enzyme and the remaining part of the hybrid biofunctional composites, i.e. RGO-PEI, ensures that the enzyme is not altered when contained inside the PEI matrices, as the enzyme does not form any bonds with the PEI matrices material or the RGO. Instead, the enzyme is allowed to move ‘freely’ inside the polymer cage to the extent that is defined by the size of the enzyme and the polymer matrix pockets.

The enzyme well retains its structures and catalytic activity because it is located in a biological compatible matrix or environment. In other words, the catalytic properties of the enzyme is maintained even though the enzyme is coupled to RGO and PEI.

The polymer PEI is further environmentally compatible and environmentally friendly, making it an environmentally better alternative than hydrazine. It is able to form covalent bonds with GO at the same time reducing GO to RGO, the latter which is an excellent conducting material. Thus, using PEI as the reducing agent, environmentally and biologically unfriendly agents like e.g. hydrazine, can be avoided.

In addition, RGO suspensions prepared by hydrazine have a limited stability ranging from a few hours to days, depending on experimental conditions. This instability problem is avoided when the reduction of GO to RGO is obtained using PEI. Thus, by the above method is obtained a general chemical route for the preparation of electrically conductive and biocompatible RGO, which can be used in biological environments.

Also disclosed herein is a hybrid biofunctional composite comprising reduced graphene oxide (RGO), polyethylenimine (PEI) and an enzyme, wherein the RGO and PEI form covalent bonds and wherein PEI forms a biocompatible matrix electrostatically encapsulating the enzyme inside the matrix. Thereby the enzyme ‘binds’ non-covalently to the RGO-supported PEI.

Also disclosed herein is a hybrid biofunctional composite according to the above and prepared by the method disclosed herein.

Herein is further disclosed an electrode-composite structure comprising hybrid biofunctional composites as described above.

### BRIEF DESCRIPTION OF THE DRAWINGS

FIG. 1 discloses a method for preparing RGO-PEI composites.

FIG. 2a shows a cuvette (left-hand side) with a solution of the as-prepared GO nanosheets and the corresponding UV spectrum of the solution (right-hand side).

FIG. 2b shows a cuvette (left-hand side) with a solution of RGO-PEI nanosheets and the corresponding UV spectrum of the solution (right-hand side).

FIG. 2c shows a cuvette (left-hand side) with a solution of RGO nanosheets reduced by hydrazine (N₂H₄) and the corresponding UV spectrum of the solution (right-hand side).

FIGS. 3a-3d show AFM images of GO (FIG. 3a), RGO reduced by N₂H₄ (FIG. 3b), RGO-PEI (FIG. 3c), and RGO-PEI/GOx composite (FIG. 3d) on mica, where GOx is glucose oxidase.

FIGS. 4a-4b show high-resolution AFM images of RGO-PEI structures on mica with different resolutions.

FIGS. 5a-5d show XPS spectra of RGO-PEI.

FIG. 6 is a table summarizing the elemental analysis of the amount for surface oxygen groups in GO, RGO-N₂H₄ and RGO-PEI by XPS.

FIG. 7a shows the FTIR spectra of GO, RGO-N₂H₄ (‘marked RGO’) and RGO-PEI.

FIG. 7b is a table listing the peak modes (given in cm⁻¹) observed for GO and RGO-PEI in FIG. 7a.

FIG. 8 is a schematic overview of the preparation of hybrid biofunctional composites.

FIG. 9a shows the UV-Vis absorption spectra of GOx before and after conjugation to RGO-PEI.

FIG. 9b shows the mass ratio of absorption of the GOx to RGO-PEI obtained from the data in FIG. 9a.

FIG. 9c is a table summarizing the results shown in FIGS. 9a and 9b.

FIG. 10a shows the UV-Vis absorption spectra of ChOx before and after the adsorption to RGO-PEI.

FIG. 10b shows the mass ratio of the absorption of ChOx to RGO-PEI shown in the UV-VIS spectra in FIG. 10a.

FIG. 10c is a list of the data analysis of the mass ratio of ChOx to RGO-PEI based on the results shown in FIGS. 10a and 10b.

FIG. 11a shows the electrocatalytic oxidation of cholesterol with different concentrations at a glassy carbon electrode (GCE) surface coated with the RGO-PEI-ChOx composite.

FIG. 11b shows a calibration plot of ChOx biosensors in response to cholesterol based on the data shown in FIG. 11a.

FIG. 12a shows the electrocatalytic oxidation of glucose at edge plane graphite electrode (EPG)/RGO-PEI/GOx.

FIG. 12b shows the amperometric response of EPG/RGO-PEI/GOx with successive addition of glucose in 10 mM PBS (pH 7.0) containing 0.8 mM Fc-COOH at 0.35 V.

FIG. 12c shows the amperometric responses versus the glucose concentration.

FIG. 12d shows a calibration plot of the biosensor for glucose based on the results shown in FIG. 12a.

FIG. 13a-b show measurements of the glucose levels in human blood samples using the hybrid biofunctional composite sensor according to the invention.

FIG. 14a-b show measurements of the concentration of glucose in human blood sample obtained from Glostrup Hospital, Denmark using the hybrid biofunctional composite sensor according to the invention.

FIG. 15 shows the long term stability of the hybrid biofunctional composites sensor according to this invention.

### DESCRIPTION OF PREFERRED EMBODIMENTS

Disclosed herein is therefore a general chemical method for preparing hybrid biofunctional composites comprising reduced graphene oxide (RGO), polyethylenimine (PEI) and an enzyme.

In one or more embodiments the enzyme has an isoelectric point below 10.

In one or more embodiments the enzyme is chosen from the group of glucose oxidase (GOx), cholesterol oxidase (ChOx), horseradish peroxidase (HRP), alcohol dehydrogenases (ADH), and Choline oxidase.

The method for preparing hybrid biofunctional composites comprising the steps of providing an aqueous solution of graphene oxide (GO), reducing the GO by adding PEI to the aqueous solution of GO thereby obtaining the aqueous RGO-PEI solution, and mixing the aqueous RGO-PEI solution with an enzyme thereby obtaining an hybrid biofunctional composite.

The RGO and the PEI form covalent bonds and PEI forms a biocompatible matrix electrostatically encapsulating the enzyme inside the matrix. More specifically, PEI normally forms a matrix positioned on the plane and edges of the RGO nanosheets, where the PEI matrix forms cages which electrostatically encapsulates the enzyme inside the matrix. Thus, the enzyme binds non-covalently to the RGO supported PEI.

In one or more embodiments, the PEI polymer has an average polymeric length of at least 60.000 monomeric units. Alternatively, the PEI polymer has an average polymeric length of at least 10.000 monomeric units or at least 25.000 monomeric units.

In one or more embodiments, the PEI polymer has monomeric units with the molecular formula

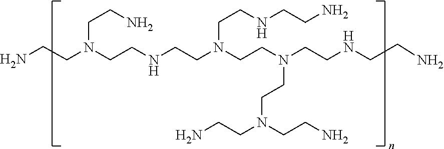

In one or more embodiments the obtained solution after PEI is added is stirred for between 30 min.-90 min., or 45 min.-75 min., or for 60 min. at a temperature between 70-120° C., or between 80-110° C., or between 90-100° C., or at 95° C.

In one or more embodiments, mixing the aqueous RGO-PEI solution with the enzyme in step is done at a temperature between 1-10° C., or between 2-8° C., or between 3-6° C., or between 4-5° C., or at 4° C. for between 6-24 hours, or between 8-18 hours, or between 10-14 hours.

In one or more embodiments the mixture obtained when mixing the aqueous RGO-PEI solution with the enzyme is centrifuged at 8000 rpm for 15 minutes after being mixed at the temperature between 1-10° C., or between 2-8° C., or between 3-6° C., or between 4-5° C., or at 4° C. for between 6-24 hours, or between 8-18 hours, or between 10-14 hours.

In one or more embodiments the method for preparing hybrid biofunctional composites further comprising the steps of washing the solution obtained when mixing the aqueous RGO-PEI solution with the enzyme with phosphate buffered saline (PBS), and successively centrifugating the solution, e.g. three times, to remove loosely bound enzymes.

The hybrid biofunctional composite produced by the above described method may in one or more embodiments be used as an enzyme-based biosensing material for a graphene based biosensors.

Alternatively, the hybrid biofunctional composite may be used for:
conjugating toxic heavy metal ions such as Pb²⁺, Hg²⁺ and Cd²⁺;
clean environmental and water technology; or
drug delivery where RGO-PEI captures and releases specific drugs.

The RGO is obtained using polyethylenimine (PEI) as both reducing agent and functional linker. PEI is a polymer with abundant amine groups, composed of ethylenimine moieties as the repeating unit. PEI is known as a highly branched, positively charged and water soluble polymer. In the past few years, PEI has received tremendous attention as versatile building blocks for the construction of adsorbents as a result of its high amine density and accessible primary amine sites on its branched chains.

The RGO-PEI material exhibited significant improvement of the biocompatibility, which could provide a microenvironment for the accommodation of different kinds of enzymes. Therefore, the biocompatibility and the excellent electron transfer properties of this RGO-PEI-enzyme hybrid material pave the way for its use in biosensing.

Similarly, GO contains oxygen functional groups on their basal planes and edges. Therefore, GO could show high affinity to amines or amine containing molecules. When PEI is attached to GO nanosheets the residual amine groups in PEI can exhibit good adsorption capacity for anionic materials, such as polyanions and negatively charged organic, inorganic and biological molecules .

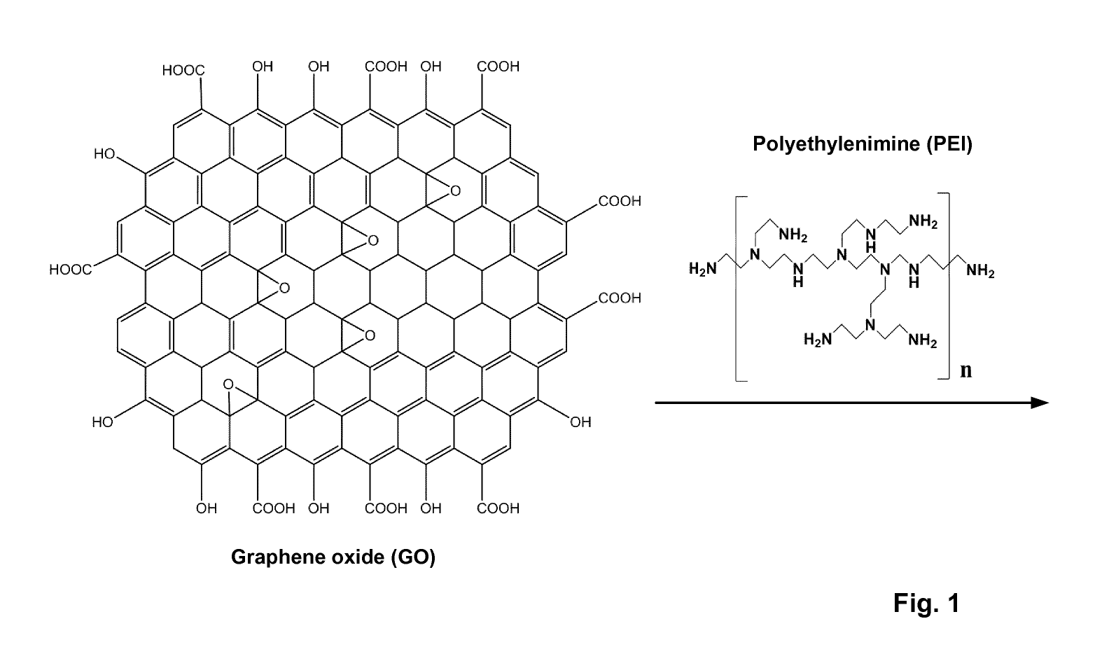

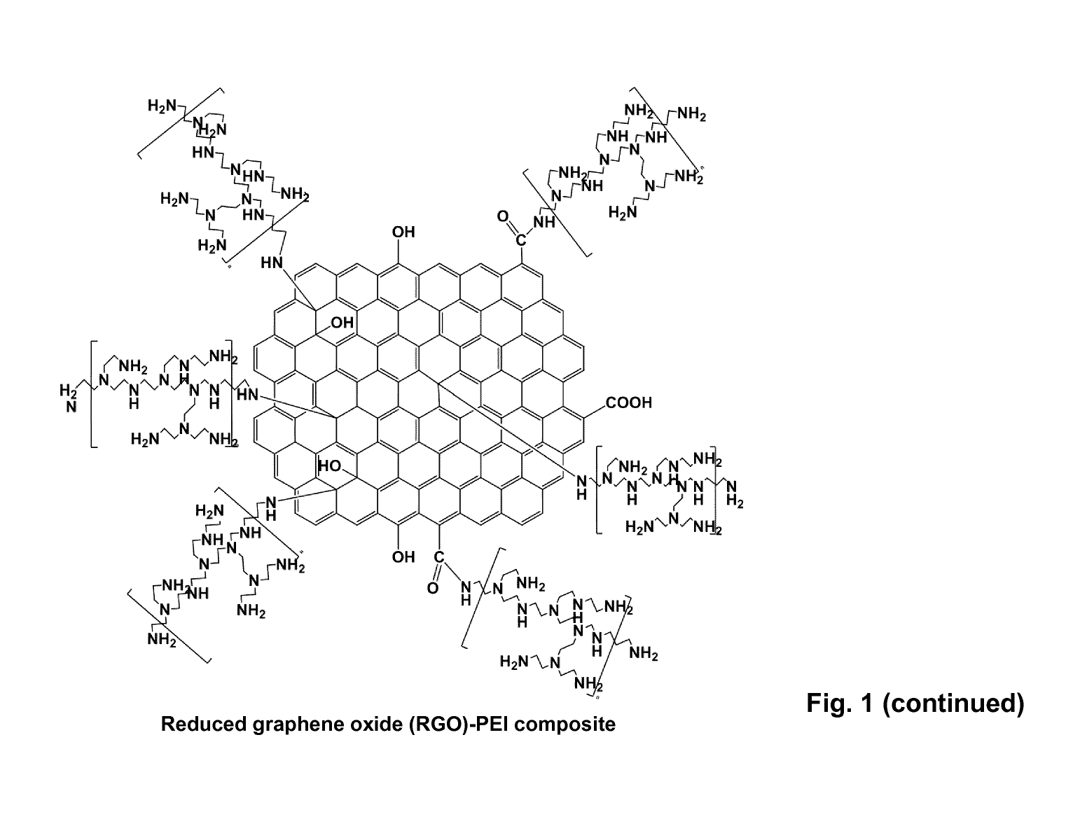

Wet-chemical synthesis of RGO based on PEI reduction is illustrated schematically in FIG. 1. A typical procedure for preparing RGO-PEI composite is conducted by mixing 400 μl 0.1 g/ml PEI with 80 ml H₂O, and then adding 20 ml 0.1 mg/ml GO. The mixed solution is normally stirred at 95° for 1 h. The change of color from brown to black indicates the reduction of GO to RGO by PEI .

The Graphene oxide (GO) is normally prepared by the modified Hummer's method with graphite flake <20 μm, used as a starting material. Preparation of graphene oxide (GO) involves a two-step process, where pre-oxidized graphite is prepared in a first step. Graphite powder (5.0 g) is slowly added into concentrated H₂SO₄ solution (7.5 ml) containing P₂O₅ (2.5 g) and K₂S₂O₈ (2.5 g) kept in a hot water bath (80° C.) under strong stirring for 3 h. After cooling to the room temperature and diluting with Milli-Q water, the dark green mixture is filtered and washed several times until waste solution pH reaching neutral. The pre-oxidized graphite powder is afterwards collected and dried in air at room temperature overnight.

In the second step, pre-oxidized graphite powder (1.0 g) is slowly added to a concentrated H₂SO₄ solution (23 ml) in an ice-water bath (0° C.). KMnO₄ (3.0 g) is then added to the mixture under slow stirring keeping the whole process below 20° C. After removing the ice-water bath, the mixture is reacted at 35° C. for 2 h with stirring and Milli-Q water (46 ml) added. After a few minutes, Milli-Q water (137.5 ml) and 2.5 ml of a 30% H₂O₂ solution are further added to the mixture, leading to the solution colour rapidly changing to bright yellow. The mixture is then washed with a 1:10 HCl solution (v/v, 250 ml) and filtered to remove residual metal ions. The raw GO suspended in Milli-Q water is centrifuged at a high rotation speed (12000 rpm min⁻¹). The supernatant containing highly dispersed and stable GO nanosheets is afterwards collected. To remove residual salts and acids, the supernatant is further dialyzed using a dialysis tube (with a cut-off molecular weight of 12000-14000) for at least one week by changing water bath regularly (2-3 times per day).

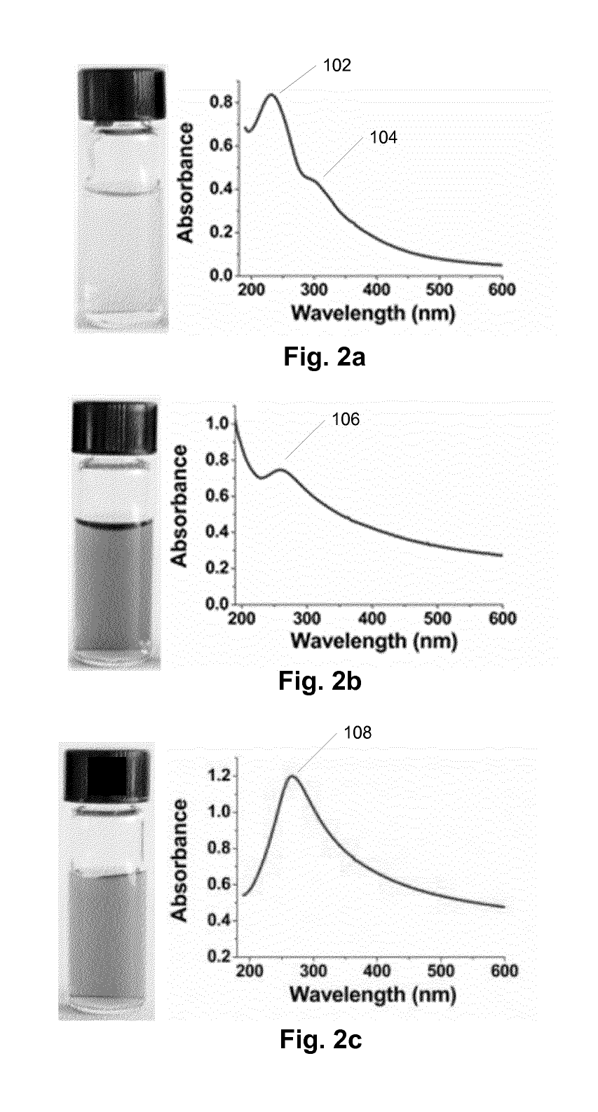

As mentioned above, during the synthesis of the RGO-PEI shown in FIG. 1, the colour of the dispersion changed gradually from brown to black over a period of approximately 60 min. FIG. 2a shows a picture (left-hand side) of a cuvette with a solution of the as-prepared GO nanosheets and the corresponding UV spectrum of the solution (right-hand side). The UV spectrum of GO shows two absorption bands at 232 nm (marked as 102) and 302 nm (marked as 104), which are typical for GO.

FIG. 2b shows a picture (left-hand side) of a cuvette with a solution of RGO-PEI nanosheets and the corresponding UV spectrum of the solution (right-hand side).

The UV spectrum of RGO-PEI shows one absorption band at 260 nm (marked as 106).

For comparison, FIG. 2c shows a picture (left-hand side) of a cuvette with a solution of RGO nanosheets reduced by hydrazine (N₂H₄) and the corresponding

UV spectrum of the solution (right-hand side). The UV spectrum of RGO-hydrazine shows one absorption band at 266 nm (marked as 108) being close to the absorption band observed in FIG. 2b for RGO-PEI.

The RGO-PEI composites are analysed systematically by atomic force microscopy (AFM), X-ray photoelectron spectroscopy (XPS), Fourier transform infrared (FTIR) spectroscopy, Raman spectroscopy, and thermo gravimetric analysis (TGA).

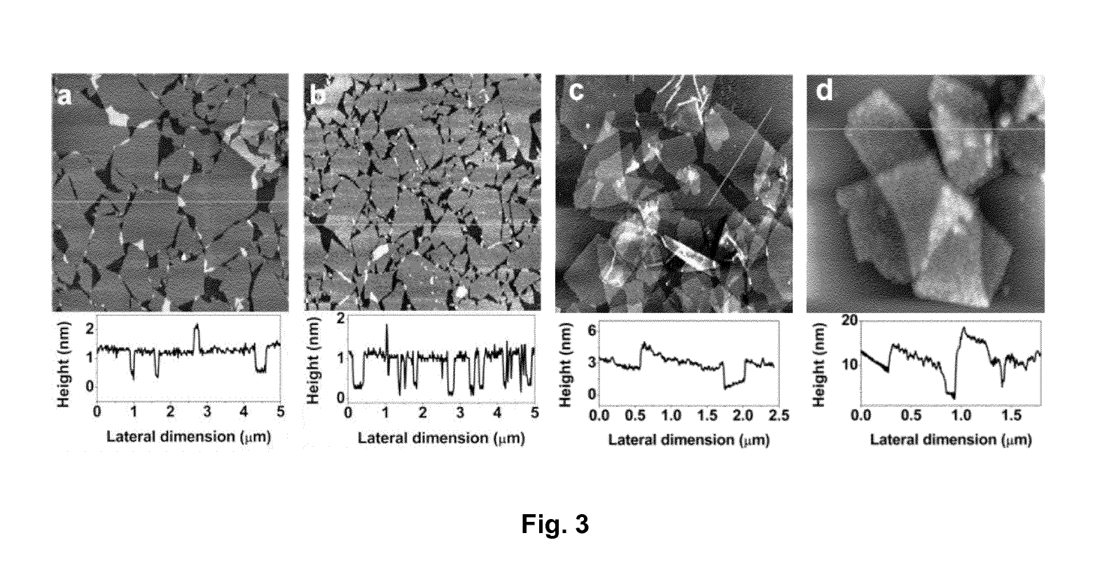

Considering the excellent dispersibility in water for the obtained RGO-PEI nanosheets, their single-sheet nature can be studied using AFM. The cross-sectional view of AFM images are shown for GO in FIG. 3a, RGO reduced by N₂H₄ in FIG. 3b, RGO-PEI in FIG. 3c, and RGO-PEI/GOx composite in FIG. 3d, where GOx is the enzyme glucose oxidase.

The dimensions in FIGS. 3a-3d are 5×5 μm² in FIG. 3a and FIG. 3b, 2.5×2.5 μm² in FIG. 3c, and 1.8×1.8 μm² in FIG. 3d. The height of GO and RGO in FIG. 3a and FIG. 3b are about 0.8 nm. The height of RGO-PEI nanosheets in FIG. 3c is 2.1˜2.5 nm, and the height of RGO-PEI/GOx composite in FIG. 3d is about 7.6˜8.3 nm.

The average thickness of a single GO sheet is found to be 0.8 nm, the average thickness of the RGO reduced by hydrazine to be 0.9 nm, and the average thickness of the RGO-PEI between 2.1 nm˜2.5 nm. The increase in average thickness of RGO-PEI compared to RGO reduced with hydrazine could be attributed to the capping reagents PEI on their surface to replace the oxygen-containing functional groups after the reduction and covalent linkage.

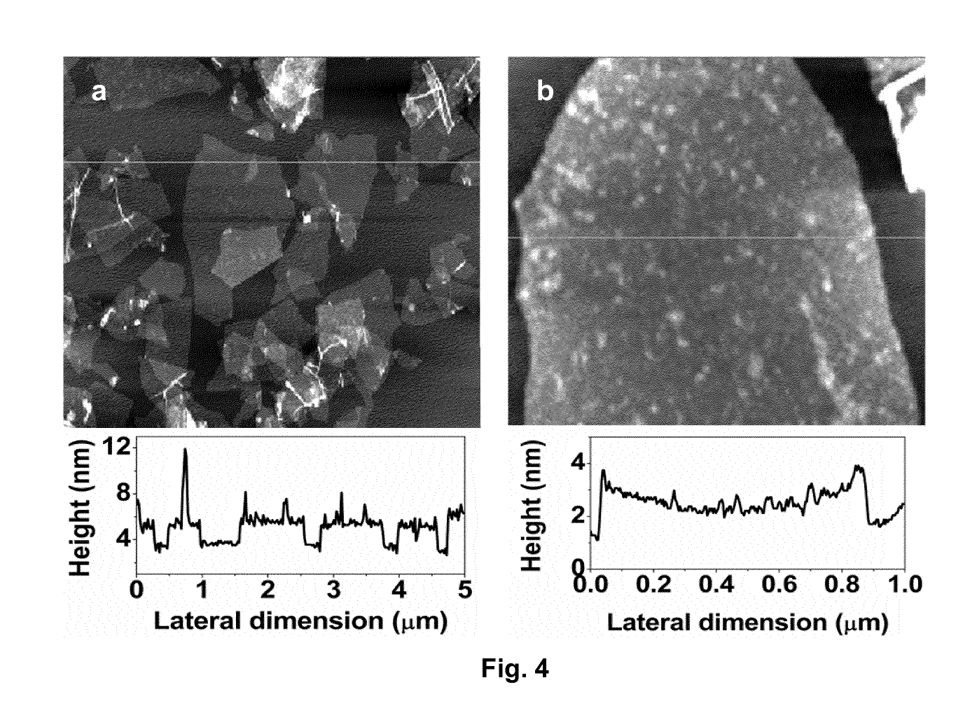

FIG. 4 shows cross-sectional views of AFM images of RGO-PEI structures on mica with a resolution of 5×5 pm² in FIG. 4a, and 1×1 μm² in FIG. 4b.

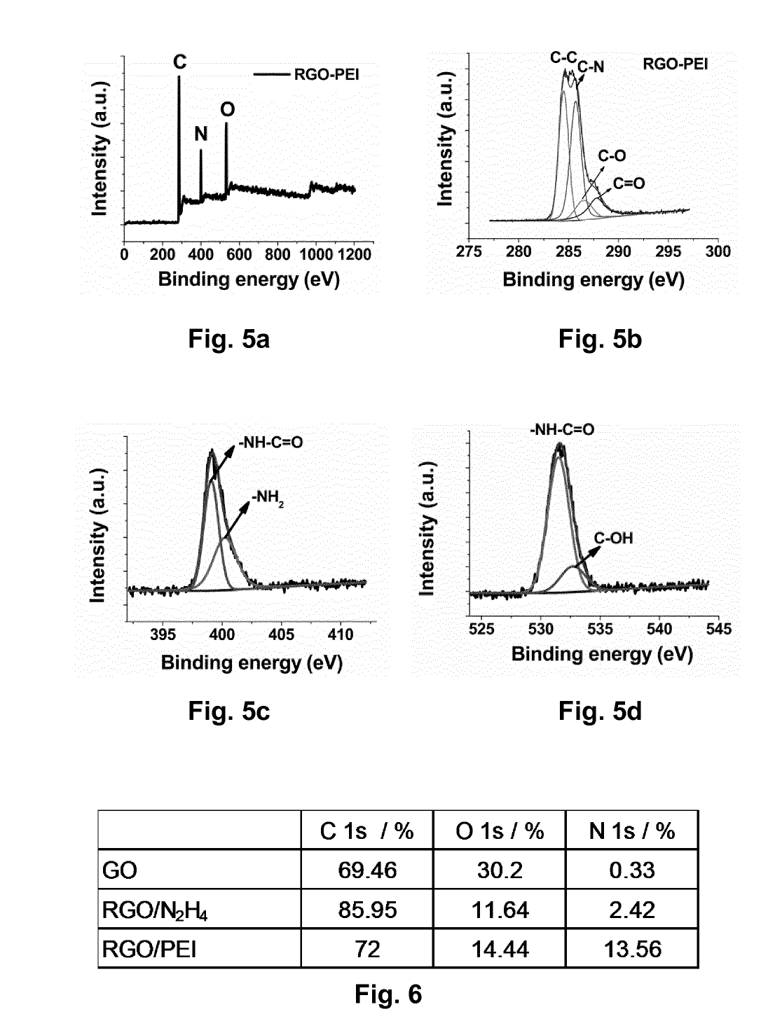

The XPS spectra of RGO-PEI is shown in FIGS. 5a-5d, where FIG. 5a shows the XPS spectrum up to 1200 eV, FIG. 5b is a close-up of the ˜285 eV peak representing the C1s, FIG. 5c is a close-up of the ˜400 eV peak representing the N1s, and FIG. 5d is a close-up of the ˜532 eV peak representing the O1s.

As shown in the table in FIG. 6, the surface oxygen groups in GO are about 30.2%. The percentage of oxygen decreases to 11.64% for RGO-N₂H₄ and similarly to 14.44% for RGO-PEI. Thus, the C/O ratio in the RGO-PEI is increased remarkably after the reduction reaction to a level similar to that observed in RGO prepared by hydrazine.

The appearance of an N peak for RGO-PEI compared to that of GO and RGO-N₂H₄ indicates the attachment of PEI onto the RGO. The N1s XPS spectra of RGO-PEI shown in FIG. 5c suggest the presence of amide (399.1 eV) and amine (400.2 eV). The O1s core-level spectrum shown in FIG. 5d can be fitted to two peaks at 531.5 eV and 532.7 eV, as is expected.

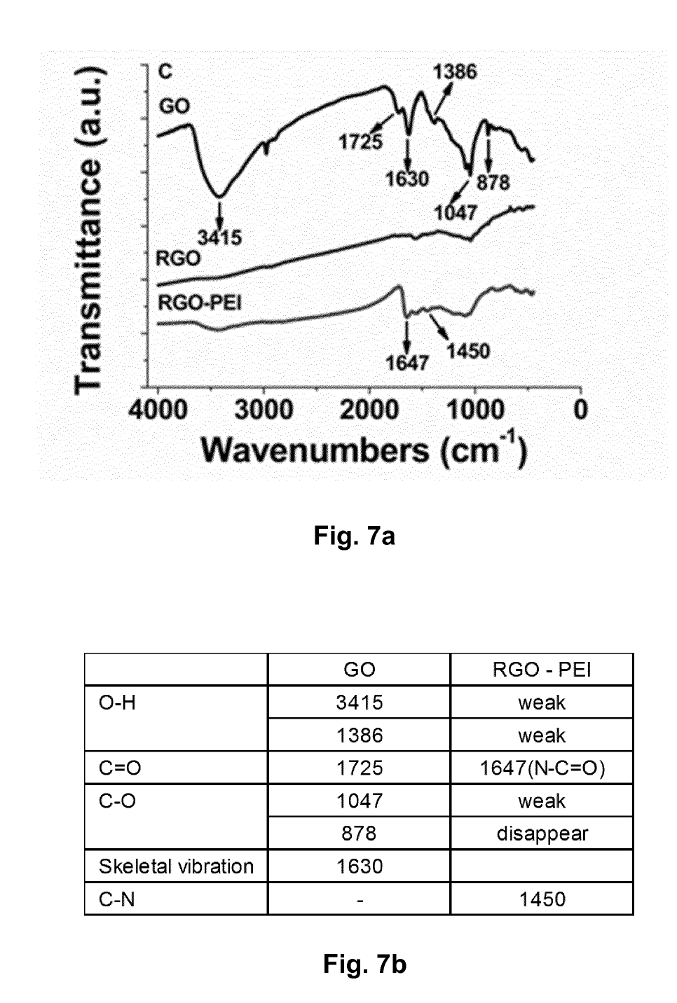

FIG. 7a shows the FTIR spectra of GO, RGO-N₂H₄ (marked RGO) and RGO-PEI and FIG. 7b is a table listing the peak modes (given in cm⁻¹) observed for GO and RGO-PEI. The O—H stretching mode and O—H bending mode are observed at 3415 cm⁻¹ and 1386 cm⁻¹, respectively, both in GO and RGO-PEI. The O—H modes are observed as a relatively strong peak in pure GO but become significantly weaker in RGO-PEI composites.

The C═O stretching in the carboxyl acids and carbonyl groups is observed at 1725 cm⁻¹ in GO, whereas it is observed at 1647 cm⁻¹ for the N—C═O group in RGO-PEI.

As a comparison, the spectrum of RGO-N₂H₄ shows a more or less flat line with a weak peak in the fingerprint region at 1047 cm⁻¹ representative of C—O stretching in the epoxy group. The same peak is observed in RGO-PEI as a weak peak and in GO as a strong peak. The 878 cm⁻¹ peak in GO is also attributed to the C—O group in the epoxy. This peak is not visible in the RGO-PEI and the RGO-N₂H₄ spectra. Skeletal vibration of graphitic domains are observed only in the GO at 1630 cm⁻¹. In RGO-PEI a weak C—N stretching is observed at 1450 cm⁻¹.

The structural characterization discussed above in connection with the preceding figures overall shows that PEI is covalently linked to the RGO nanosheets to form a biocompatible matrix. The PEI matrix therefore provides biocompatible microenvironments for accommodation of enzymes through electrostatic encapsulation. The RGO-PEI-enzyme nanocomposites can be cast into thin films on electrode surfaces, whereby the enzymes retains their catalytic activity. Thus, the resulting RGO-PEI materials described above provide a large electrochemically active surface for the adsorption of high amount of enzymes which can be used for highly sensitive and selective biosensors.

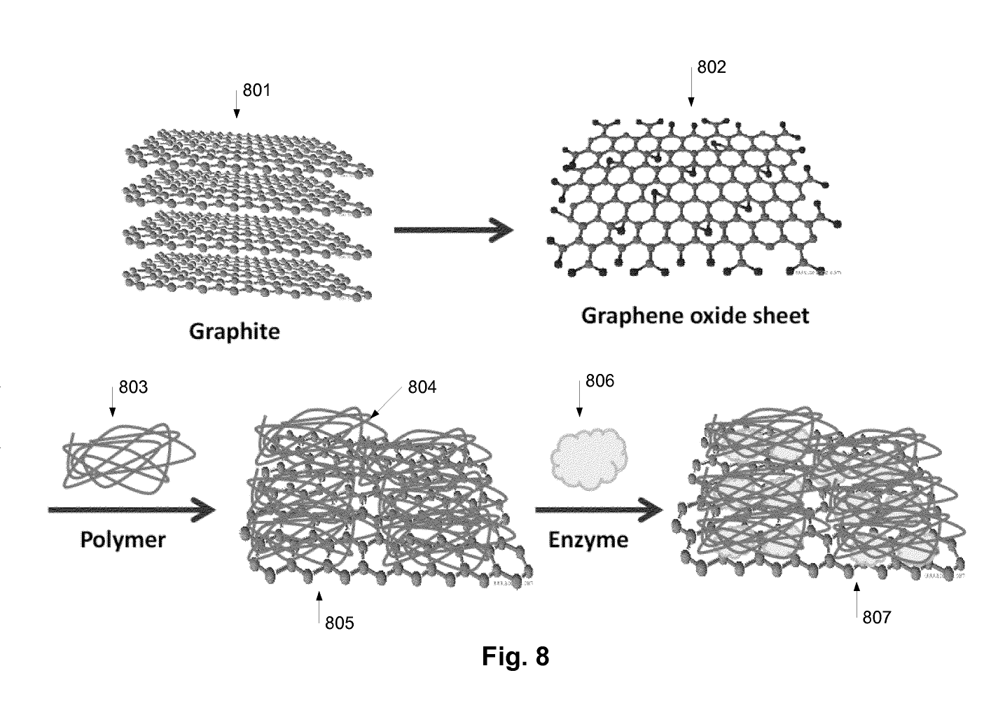

FIG. 8 is a schematic overview of the preparation of hybrid biofunctional composites comprising reduced graphene oxide (RGO), polyethylenimine (PEI) and an enzyme from here referred to as the RGO-PEI-enzyme hybrid material. In the first step in the top part, the initial synthesis of the graphene oxide sheets 802 starting from graphite 801 is illustrated. This process may be performed using the modified Hummer's method as described previously in connection with FIG. 1.

In the next step shown lower part of FIG. 8, graphene oxide is simultaneously reduced and functionalized by the polymer PEI 803 to obtain RGO-PEI 805 as also described in connection with FIG. 1. The reduction/functionalization is followed by a loading of the enzyme 806 over the RGO-PEI matrix 805 to obtain the RGO-PEI-enzyme hybrid material 807.

The RGO-PEI-enzyme hybrid composites 807 may be obtained by mixing 800 μl 0.05 mg/ml RGO-PEI with 200 μl 1 mg/ml enzyme at 4° C. overnight thereby forming RGO-PEI-enzyme hybrid composites. The mixture is afterwards centrifuged at 8000 rpm for 15 minutes and the supernatant of the solution is collected for the determination of enzyme loading capacity over the RGO-PEI matrix.

The precipitate is collected and is normally washed with phosphate buffered saline (PBS) and successively centrifuged three times to remove loosely bound enzymes from RGO-PEI matrix. The immobilization efficiency of different enzymes may be determined indirectly by the UV absorption spectra by measuring the absorption of the free amount of enzyme in the supernatant and absorption of the actual amount of enzyme added before.

As illustrated in FIG. 8, PEI forms matrix-like cages/matrices 804 on the planes and at the edges of the GO sheets in the RGO-PEI material. When adding the enzyme 806, these PEI cages 804 facilitate an accommodation for the enzyme 806 such that the enzyme 806 is encapsulated electrostatically inside the PEI-formed matrix 804. Thereby the enzyme 806 does not form any covalent bonds to either the RGO material 802 or the PEI polymer 803 in the PEI matrices 804.

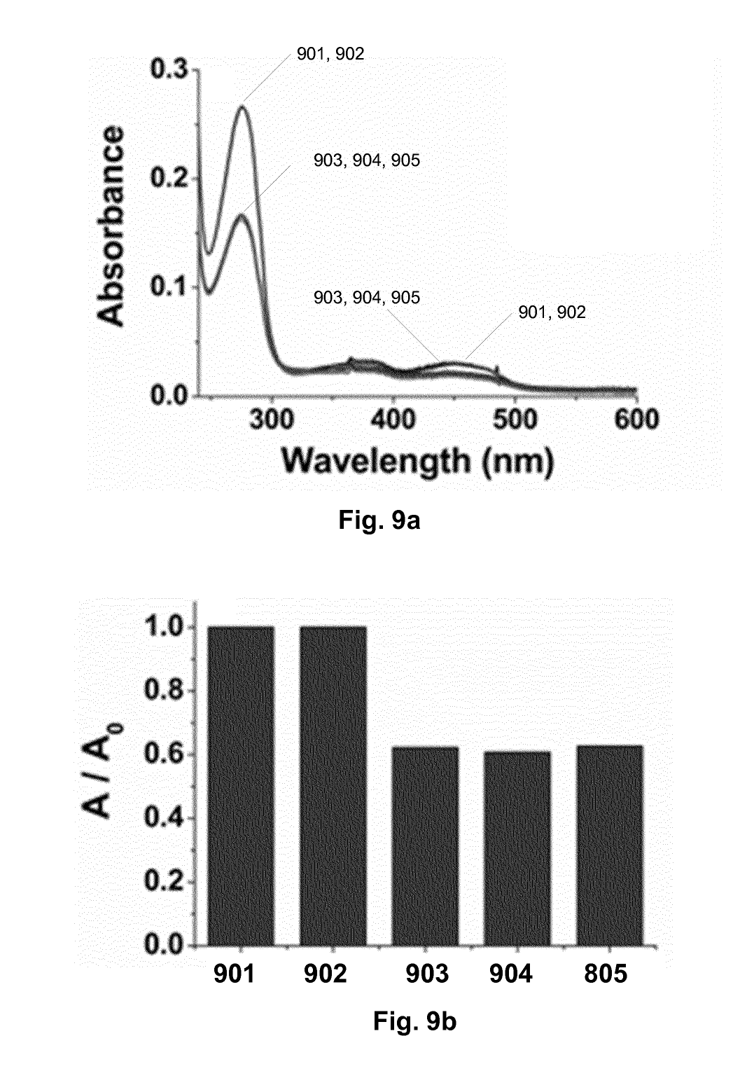

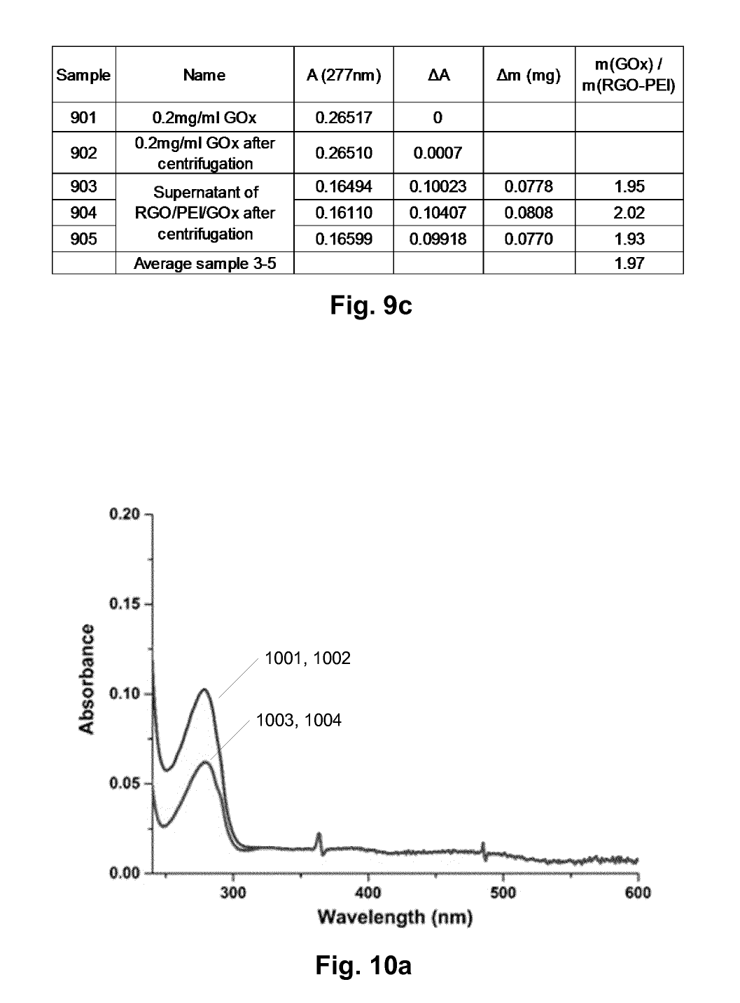

FIGS. 9-12 show further experimental data of two representative examples of enzymes accommodated in the RGO-PEI matrix; glucose oxidase (GOx) and cholesterol oxidase (ChOx) enzyme. Glucose and cholesterol are two crucial constituents of all human cells, and determination of glucose and cholesterol levels in blood is a crucial step for controlling and early diagnosis of many life threatening diseases such as diabetes and obesity.

FIG. 9a shows the UV-Vis absorption spectra of GOx before and after the adsorption to the RGO-PEI matrix. Lines 901 and 902 show that the UV-Vis absorption spectra of pure GOx before (line 901) and after (line 902) centrifugation in the absence of RGO-PEI. As seen in the figure, it is apparent that these lines more or less are completely overlapping with one another, indicating that centrifugation alone does not decrease the enzyme amount in the solution and thus this is a good control experiment. Lines 903, 904 and 905 show the UV-Vis absorption spectra of GOx after centrifugation with RGO-PEI/GOx measured for three independent samples with good reproducibility as indicated by these lines overlapping each other.

FIG. 9b shows the mass ratio of the absorption of GOx to RGO-PEI/GOx solutions shown in the UV-VIS spectra in FIG. 9a after centrifugation and compared to the original GOx solution, where A and A₀ are the absorption of the solutions at 277 nm. As can be seen, the first two samples 901, 902 representing GOx shows a 1:1 ratio between the absorption before and after centrifugation of GOx, whereas a drop to 60% of the absorption compared to GOx is observed for the RGO-PEI/GOx samples 903, 904 and 905.

FIG. 9c is a table summarizing the results shown in FIGS. 9a and 9b. The high loading of enzyme GOx with a mass ratio of about 2 is achieved.

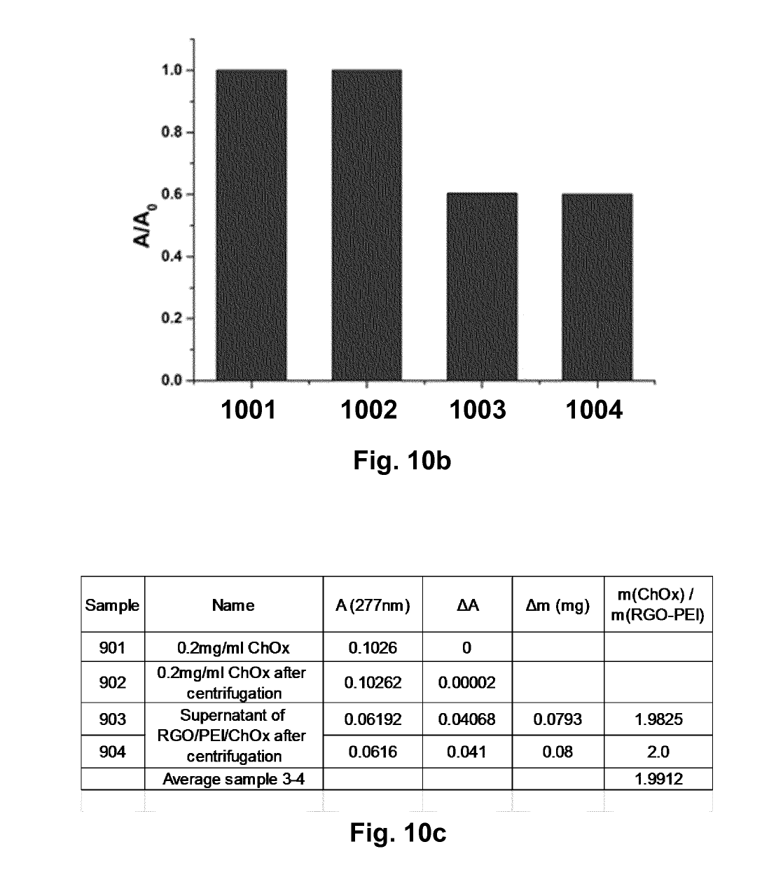

FIG. 10a shows the UV-Vis absorption spectra of ChOx in line 1001, the RGO-PEI/ChOx spectrum after centrifugation in line 1002, and the RGO-PEI/ChOx spectrum and 12 hour after centrifugation in lines 1003 and 1004.

FIG. 10b shows the ratio of absorption of the ChOx and RGO-PEI/ChOx solutions shown in the UV-VIS spectra in FIG. 10a after centrifugation and compared to the original ChOx solution, where A and A₀ are the absorption of the solutions at 277 nm. As can be seen, the first two samples 1001, 1002 representing ChOx shows a 1:1 ratio between the absorption before and after centrifugation of ChOx, whereas a drop to 60% of the absorption compared to ChOx is observed for the RGO-PEI/ChOx samples 1003 and 1004.

FIG. 10c list of the data analysis of the mass ratio of ChOx to RGO-PEI based on the results shown in FIGS. 10a and 10b. Similar to GOx, for ChOx a high loading with the mass ratio of about 2 is accomplished as well.

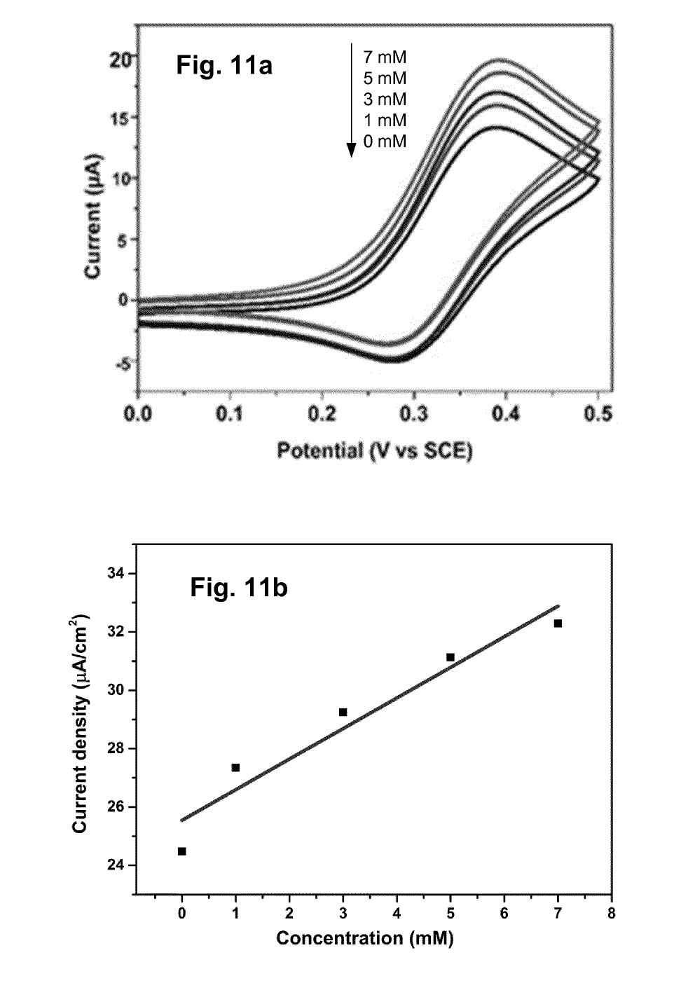

FIG. 11a shows the electrocatalytic oxidation of cholesterol at a glassy carbon electrode (GCE) surface modified with RGO (GCE/RGO/ChOx) in 10 mM PBS (pH 7.0) in the presence of 0.8 mM ferrocenecarboxylic acid (Fc-COOH) as an electrochemical mediator. A scan rate of 50 mV/s is used in all the experiments, where the concentration of cholesterol is varied from 0 mM to 7 mM as shown in FIG. 11a.

FIG. 11b shows a calibration plot (the line) of the biosensor for cholesterol based on the results shown in FIG. 11a (squares).

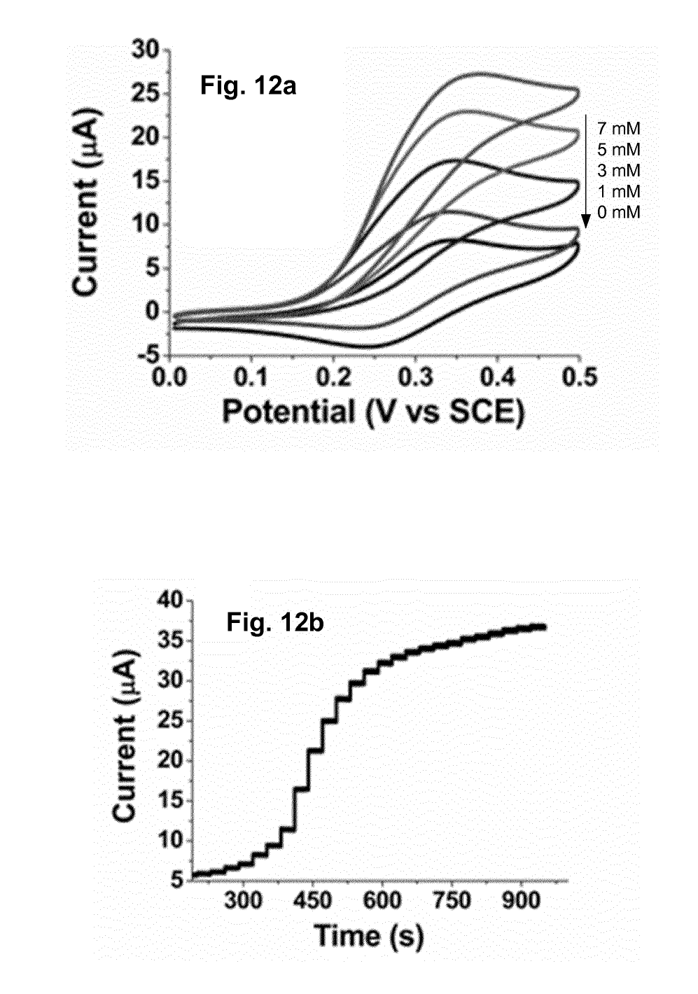

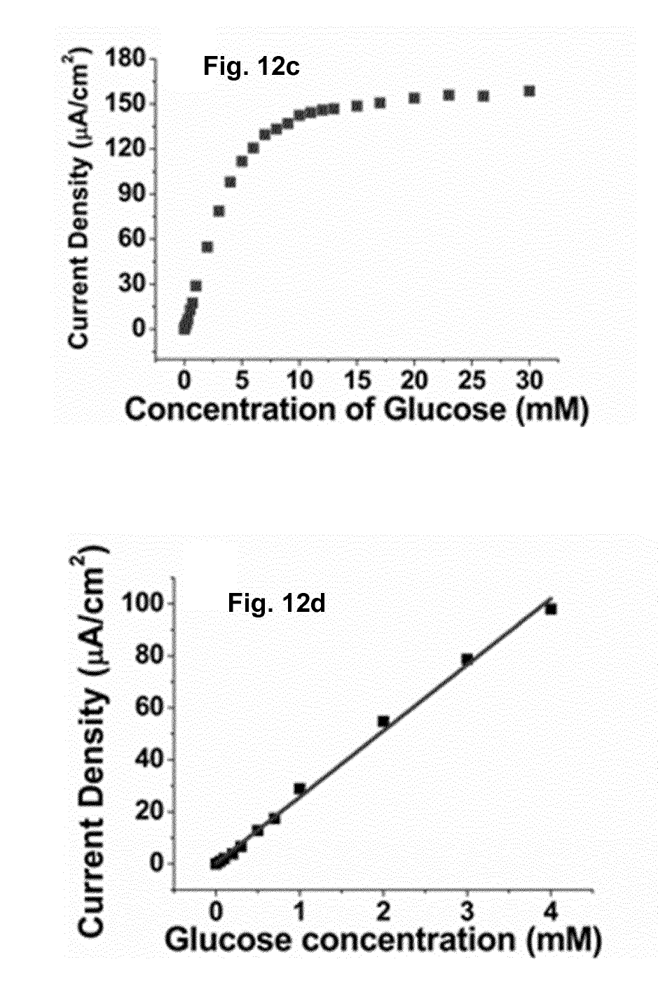

FIG. 12a shows the electrocatalytic oxidation of glucose at edge plane graphite electrode (EPG)/RGO-PEI/GOx in 10 mM PBS (pH 7.0) in the presence of 0.8 mM Fc-COOH as an electrochemical mediator. A scan rate of 10 mV/s is used in all the experiments, where the concentration of glucose is varied from 0 mM to 7 mM as shown in FIG. 12a.

FIG. 12b shows the typical amperometric response of EPG/RGO-PEI/GOx with successive addition of glucose in 10 mM PBS (pH 7.0) containing 0.8 mM Fc-COOH at 0.35 V. The amperometric responses versus the glucose concentration is shown in FIG. 12c and the calibration plot of the biosensor for glucose based on the results shown in FIG. 12a (squares) are shown as the line in FIG. 12d.

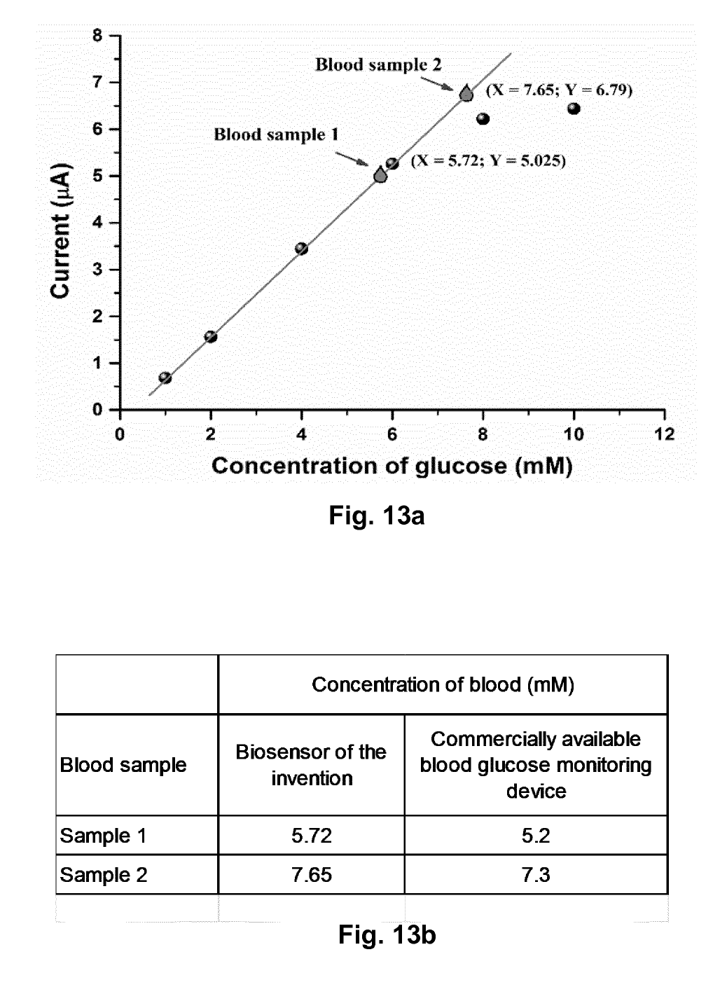

FIG. 13a shows measurements of the glucose levels in human blood samples using the hybrid biofunctional composite sensor according to the invention. The round dots corresponds to datapoints obtained from measuring the different concentrations of standard glucose solutions and the blood drops corresponds to the datapoints obtained from measuring the glucose level of the two different blood samples by using the hybrid biofunctional composite sensor.

In FIG. 13b, a table is shown a comparison of the blood glucose detection method using the hybrid biofunctional composites sensor of this invention and a commercially available blood glucose monitoring device.

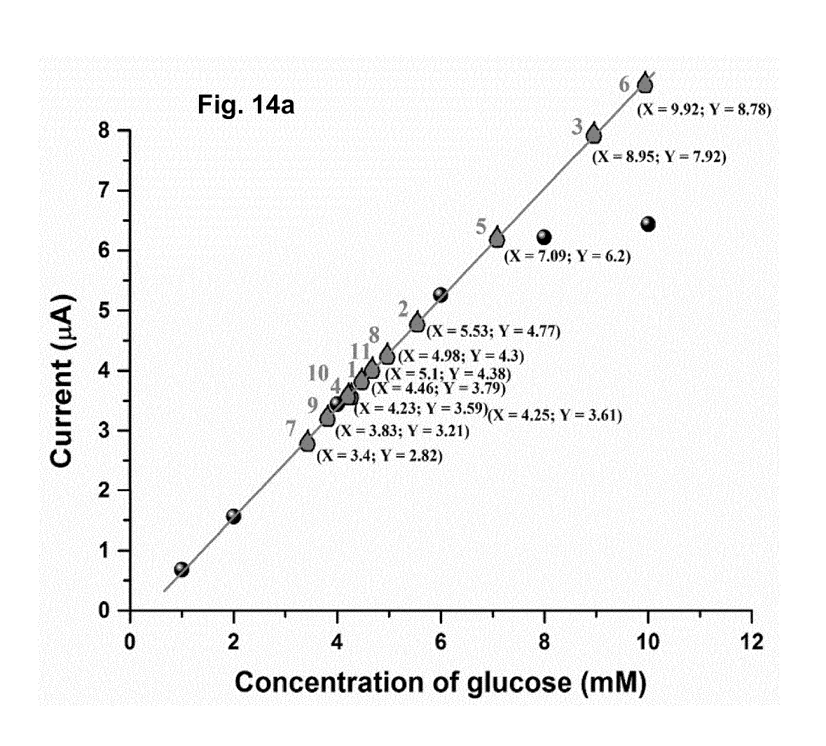

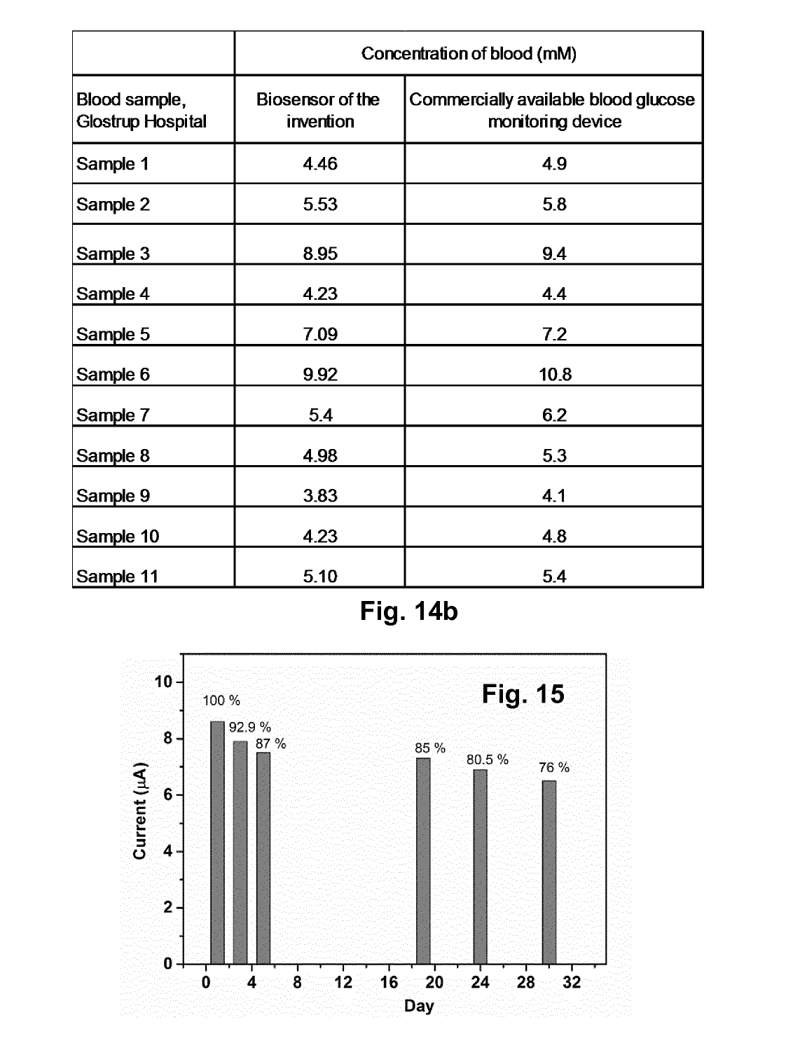

FIG. 14a shows measurements of the concentration of glucose in human blood sample obtained from Glostrup hospital, Denmark by using the hybrid biofunctional composite sensor according to the invention. The round dots corresponds to datapoint obtained from measuring the different concentrations of standard glucose solutions and the blood drops corresponds to the datapoint obtained from measuring the glucose level of eleven different blood samples (supplied by Glostrup hospital, Denmark) by using the hybrid biofunctional composite sensor.

In FIG. 14b, a table is shown a comparison of the blood glucose detection method using the hybrid biofunctional composites sensor of this invention and a commercially available blood glucose monitoring device.

FIG. 15 shows the long term stability of the hybrid biofunctional composites sensor of this invention for 30 days at 35° C. Amperometric measurements were performed with the biosensor for a period of 30 days during which the sensors were stored at 35° C. These conditions mimics the practical Summer conditions in some countries such as India and some south parts of China. The graph in FIG. 15 demonstrates that the stability is high even at very warm and humid weather conditions.

Chemicals and Materials.

Graphite flakes (<20 μm, synthetic), D-(+)-glucose 99%), and glucose oxidase (GOx, from Aspergillus niger, 100,000-250,000 units/g solid) were purchased from Sigma-Aldrich. Ferrocenecarboxylic acid (97.0% (Fe)), poly(ethylenimine) solution (50% (w/v) in water, M_{w}=750,000), K₂HPO₄ and KH₂PO₄ were purchased from Fluka. Phosphate buffer solutions (PBS) were employed as supporting electrolyte and the pH value was adjusted to 7.0 with K₂HPO₄ and KH₂PO₄. All chemicals were used as received. All solutions were prepared with Milli-Q water (18.2 Me).

Instruments

The UV-vis spectra were recorded using a single-beam spectrophotometer (HP8453, Hewlett Packard).

Atomic force microscopy (AFM) imaging was performed in the tapping mode using a 5500AFM system (Agilent Technologies, Chandler, USA).

X-ray photoelectron spectroscopy (XPS) analysis was carried out by an ESCALABMKII X-ray photoelectron spectrometer.

Fourier transform infrared spectra (FTIR) were recorded in the solid state using KBr substrates containing the target materials by a Perkin Elmer Spectrum.

Thermo gravimetric analysis (TGA, Netzsch STA 409PC) was reported in an N₂ atmosphere at a heating rate of 5° C. min⁻¹. A CHI 760C (USA) and an Autolab (Eco Chemie, The Netherlands) instrument in combination with a three-electrode system were used for electrochemical experiments. An edge plane graphite (EPG, d=5 mm), a bright Pt wire and a saturated calomel electrode (SCE) were used as working electrode, counter electrode, and reference electrode, respectively. Electrolyte solutions were deoxygenated for 30 mins by Ar purified by Chrompack (oxygen<50 ppb). All systems were blanketed with an Ar-atmosphere during measurements.

The EPG was freshly cleaned by polishing on 1.0 μm, 0.3 μm, 0.05 μm Al₂O₃ slurry (Electron Microscopy Science, PA, USA) followed by ultra-sonication in Millipore water. Then the RGO-PEI-enzyme hybrid material was drop casted on the electrode surface for further electrochemical characterization.

1. A method for preparing hybrid biofunctional composites comprising reduced graphene oxide (RGO), polyethylenimine (PEI) and an enzyme, the method comprising:
A) providing an aqueous solution of graphene oxide (GO);
B) reducing the GO by adding PEI to the aqueous solution of GO thereby obtaining an aqueous RGO-PEI solution; and
C) mixing the aqueous RGO-PEI solution with an enzyme thereby obtaining the hybrid biofunctional composite,
wherein the RGO and the PEI form covalent bonds and PEI forms a biocompatible matrix electrostatically encapsulating the enzyme inside the matrix.

2-17. (canceled)

18. The method for preparing hybrid biofunctional composites according to claim 1, wherein the enzyme has an isoelectric point below 10.

19. The method for preparing hybrid biofunctional composites according to claim 1, wherein the enzyme is glucose oxidase (GOx), cholesterol oxidase (ChOx), horseradish peroxidase (HRP), alcohol dehydrogenases (ADH), or Choline oxidase.

20. The method for preparing hybrid biofunctional composites according to claim 1, wherein the PEI polymer has an average polymeric length of at least 60.000 monomeric units.

21. The method for preparing hybrid biofunctional composites according to claim 1, wherein the PEI polymer has monomeric units with the molecular formula:

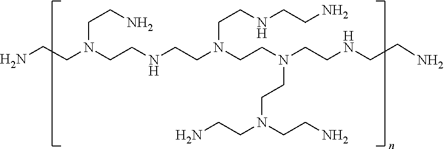

22. The method for preparing hybrid biofunctional composites according to claim 1, wherein after PEI is added in step B), the obtained solution is stirred for between 30 min.-90 min., or 45 min.-75 min., or for 60 min. at a temperature between 70-120° C., or between 80-110° C., or between 90-100° C., or at 95° C.

23. The method for preparing hybrid biofunctional composites according to claim 1, wherein mixing the aqueous RGO-PEI solution with the enzyme in step C) is done at a temperature between 1-10° C., or between 2-8° C., or between 3-6° C., or between 4-5° C., or at 4° C. for between 6-24 hours, or between 8-18 hours, or between 10-14 hours.

24. The method for preparing hybrid biofunctional composites according to claim 22, wherein the mixture obtained in step C) is centrifuged at 8000 rpm for 15 minutes after being mixed at the temperature between 1-10° C., or between 2-8° C., or between 3-6° C., or between 4-5° C., or at 4° C. for between 6-24 hours, or between 8-18 hours, or between 10-14 hours.

25. The method for preparing hybrid biofunctional composites according to claim 1 further comprising:
D) washing the obtained solution in step C) with phosphate buffered saline (PBS), and
E) successively centrifugating the solutionto remove loosely bound enzymes.

26. A hybrid biofunctional composite comprising reduced graphene oxide (RGO), polyethylenimine (PEI) and an enzyme, wherein the RGO and PEI form covalent bonds and wherein PEI forms a biocompatible matrix electrostatically encapsulating the enzyme inside the matrix.

27. The hybrid biofunctional composite according to claim 26, wherein the hybrid biofunctional composite is prepared by the method for preparing hybrid biofunctional composites according to claim 1.

28. The hybrid biofunctional composite according to claim 26, wherein the enzyme has an isoelectric point below 10 and/or is chosen from the group of glucose oxidase (GOx), cholesterol oxidase (ChOx), horseradish peroxidase (HRP), alcohol dehydrogenases (ADH), and Choline oxidase.

29. The hybrid biofunctional composites according to claim 26, wherein the PEI has an average polymeric length of at least 60.000 monomeric units.

30. The hybrid biofunctional composites according to claim 26, wherein the PEI polymer has monomeric units with the molecular formula:

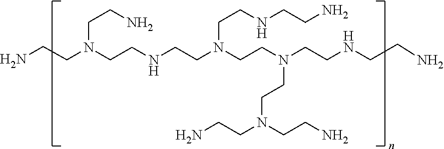

31. A method of using the hybrid biofunctional composite of claim 1 to detect blood glucose comprising:
contacting the hybrid biofunctional composite of claim 1 with a blood sample; and
measuring the level of glucose in said blood sample.

32. A method of using the hybrid biofunctional composite of claim 1 comprising:
providing the hybrid biofunctional composite of claim 1; and
conjugating Pb²⁺, Hg²⁺ and Cd²⁺ or
delivering a drug whereby the RGO-PEI captures and releases the drug.

33. An electrode-composite structure comprising a hybrid biofunctional composites according to claim 26.
